(19) United States
(12) Patent Application Publication
(10) Pub. No.: US 2021/0391062 A1
(43) Pub. Date: Dec. 16, 2021

(54) SYSTEMS AND METHODS FOR PREDICTING LENGTH-OF-STAY WITH AI

(71) Applicant: TAGNOS, INC., Irvine, CA (US)

(72) Inventor: Neeraj BHAVANI, Aliso Viejo, CA (US)

(21) Appl. No.: 16/899,296

(22) Filed: Jun. 11, 2020

(51) Int. Cl. G16H 40/20; G16H 50/70; G16H 50/30; G06N 20/00

(52) U.S. Cl. CPC G16H 40/20; G06N 20/00; G16H 50/30; G16H 50/70

(57) ABSTRACT

Systems and methods are disclosed for an AI learning system to predict patient length of stay (LOS) in healthcare facilities. The AI learning systems uses historic data from a healthcare facility and demographic data local to the facility to develop a predictive model for LOS for a patient. The system uses the contemporaneous data from the healthcare facility related to patient treatment and healthcare resources to predict LOS continuously from patient admittance to discharge. Patients with predicted LOS greater than an insurance reimbursed maximum are flagged as at risk and blocking factors are identified. The system recommends proactive steps to resolve the blocking factors and reduce the LOS for at risk patients. The system self modifies the model to minimize the delta between predicted LOS and actual LOS.

### FIELD OF THE INVENTION

The field of the invention is healthcare facilities and activities.

### BACKGROUND

Managing, orchestrating, or optimizing patient care or healthcare events is a substantial problem for large facilities or enterprises, and as such an area of great interest and development. However, monitoring and accurately predicting resource requirements or availability is incredibly complicated and often yields suboptimal or unacceptable results. Such complications are compounded in industries where multiple, concurrent operations are performed that require overlapping resources with shared upstream or downstream tasks, all occurring within a confined space (e.g., manufacturing facilities, construction sites, healthcare facilities, etc). Further compounding the problem healthcare facilities is health insurance or Medicare often provide limited financial coverage, resulting in the healthcare facility taking a financial loss if resource allocations are beyond the limit.

For example, hospital executives today are constantly monitoring the Length-of-Stay (LOS) of the patient inside the hospital to improve clinical outcomes and optimize that against the standardized payments they receive from Medicare and other payors. In other words, every patient has a definite number of days a hospital will get paid for based on the DRG (Diagnosis Related Groups) code. Another incentive for hospitals to optimize LOS is to reduce their liability risk on exposure to Hospital Acquired Infection (HAI).

Currently, Chief Medical Officer and Case Managers typically have daily huddles to improve patient care and patient flow. In addition, hospital Chief Financial Officers (CFOs) constantly try to optimize staffing hours based on patient census. This helps to plan-ahead on logistics and demand for room and bed allocation.

All publications referenced herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

There is an unfulfilled need for improved systems, methods, or solutions for monitoring occupancy of a healthcare facility, including identifying patients that will break-even on LOS, patients that may exceed LOS without preventative steps, and patients that will exceed LOS and require mitigation steps. There is also a need for solutions that identify patients at risk of exceeding LOS upon admittance to the healthcare facility. There is also a need to automatically identify and monitor clinical indicators as well as changing diagnosis that change the LOS or risk of a patient exceeding the LOS. There is also a need to monitor and assess discharge risk factors at a high level. There is a further need to monitor community or demographic data in an effort to predict expected healthcare traffic or traffic to specific departments.

Thus, there remains a need for methods and systems to continually improve prediction of resource requirements and availability at health care facility in view of limitations placed on resources.

### SUMMARY OF THE INVENTION

The inventive subject matter contemplates devices, systems, and methods for implementing a resource management system in a healthcare facility. A first health data is used to develop a model to predict a first resource requirement in the healthcare facility. A predictive ability/capacity of the model is assessed by applying a second health data to the model to predict a second resource requirement, and comparing the predicted second resource requirement to a known resource requirement. The model is informationally coupled with a database of health data for the healthcare facility. The database includes contemporaneous data from the healthcare facility, preferably updated on a live basis. The resource management system employing the model is then implemented in the healthcare facility. Preferably, the model continually evolves to improve prediction of resource requirements and identify factors affecting resource requirements.

Resource management systems for a healthcare facility are further contemplated. A predictive element (e.g., processor, machine learning algorithm (MLA), etc.) is informationally coupled to a database of health data regarding the healthcare facility, and a health data generator is coupled to the database. The health data generator provides a class of contemporaneous health data to the database, and the predictive element uses the database to make a model to predict a health event in the healthcare facility based on the class of contemporaneous health data.

Further methods of improving resource management in a healthcare facility are contemplated. A model is developed to predict a healthcare event in the healthcare facility. Contemporaneous data regarding the healthcare facility is accessed, and the model is applied to the contemporaneous data to predict the healthcare event. A resource responsive to the healthcare event is then allocated.

Various objects, features, aspects and advantages of the inventive subject matter will become more apparent from the detailed description of preferred embodiments, along with the accompanying drawing figures in which like numerals represent like components.

### BRIEF DESCRIPTION OF THE DRAWING

FIG. 1 depicts a system of the inventive subject matter.

FIG. 2 depicts a user interface of the inventive subject matter.

FIG. 3 depicts report interface of the inventive subject matter.

### DETAILED DESCRIPTION

The inventive subject matter contemplates devices, systems, and methods for implementing a resource management system in a healthcare facility. A first health data is used to develop a model to predict a first resource requirement in the healthcare facility. A predictive ability/capacity of the model is assessed by applying a second health data to the model to predict a second resource requirement, and comparing the predicted second resource requirement to a known resource requirement. The model is informationally coupled with a database of health data for the healthcare facility. The database includes contemporaneous data from the healthcare facility, preferably updated on a live basis. The resource management system employing the model is then implemented in the healthcare facility. Preferably, the model continually evolves to improve prediction of resource requirements and identify factors affecting resource requirements.

The model preferably includes or is developed using a machine learning algorithm (MLA) or other artificial intelligence (AI) form factor. The machine learning algorithm employs at least one of supervised, unsupervised, reinforcement, or ensemble learning methodology, or combinations thereof. The model is preferably applied to contemporaneous data from the healthcare facility to predict an unknown of unquantified resource requirement at the healthcare facility, for example at least one of LOS of a patient, equipment required for a patient, or personnel required for a patient. The MLA typically self modifies to minimize a delta between a predicted resource requirement and an actual resource requirement on a contemporaneous basis, preferably reducing the delta to less than 20%, more preferably less than 10%, and more preferably less than 5% or 3%.

In some embodiments, the model further identifies a class or classes of health data that affects a predicted outcome of the model by more than 5%, 10%, 15%, 20%, 25%, 30%, or 40%. Moreover, the model identifies classes of health data that, when combined simultaneously or sequentially, have a synergistic or reduced affect on the predicted outcome. It is contemplated that the model self modifies to weight various classes of data in order to achieve the lowest delta between predicted and actual outcome. The database includes classes of data related to at least one of an operational outcome of the healthcare facility, a financial outcome of the healthcare facility, a diagnosis of a patient, a prognosis of a patient, a patient outcome, or a treatment of a patient, and can further include demographic data regarding one or more patients, as well as demographic, health, social, cultural, regulatory, or recent event data for the community, county, or region local to the healthcare facility.

In some embodiments, the resource management system further identifies whether a predicted resource requirement exceeds a limit (e.g., LOS, capacity, available quantity, limit of insurance coverage, etc.). The resource management system preferably provides a recommended act to minimize a delta between a predicted resource requirement and a limit. The resource management system also assigns a risk index to a patient based on a delta between a predicted resource requirement and a limit with respect to that patient. For example, where a patient has a predicted LOS greater (e.g., more than 1, 2, 3, 4, or 5 days, etc.) than insurance will cover, that patient is assigned a high risk index. Similarly, patients with a predicted LOS equal to or close to the limit covered by insurance (e.g., less than 1 or 2 days more, etc.) are assigned a medium risk index, while patients with a predicted LOS equal to or less than the insurance limit have are assigned a low risk index. Additional resources may be diverted to patient's with a high risk index to mitigate or avoid exceeding insurance limits.

Resource management systems for a healthcare facility are further contemplated. A predictive element (e.g., processor, machine learning algorithm (MLA), etc.) is informationally coupled to a database of health data regarding the healthcare facility, and a health data generator is coupled to the database. The health data generator provides a class of contemporaneous health data to the database, and the predictive element uses the database to make a model to predict a health event in the healthcare facility based on the class of contemporaneous health data.

The model typically includes or is developed by a machine learning algorithm that employs at least one of supervised, unsupervised, reinforcement, or ensemble learning methodology. The predicted health event is typically at least one of LOS, required equipment, or required personnel for a patient. Classes of contemporaneous health data typically relate to at least one of an operational outcome of the healthcare facility, a financial outcome of the healthcare facility, a diagnosis of a patient, a prognosis of a patient, a patient outcome, or a treatment of a patient, but additional data such as demographic data for patients or demographic data, health data, social data, cultural data, regulatory data, or recent event data for the community, county, or region local to the healthcare facility can also be included.

Preferably the machine learning algorithm self modifies to minimize a delta between a predicted health event and an actual health event. The resource management system typically provides a recommended act to minimize a delta between a predicted health event and a proscribed limitation.

Further methods of improving resource management in a healthcare facility are contemplated. A model is developed to predict a healthcare event in the healthcare facility. Contemporaneous data regarding the healthcare facility is accessed, and the model is applied to the contemporaneous data to predict the healthcare event. A resource responsive to the healthcare event is then allocated. The model typically includes or is developed by a machine learning algorithm trained on historic data related to the healthcare facility, comparable health care facilities, the healthcare event, or comparable healthcare events. Preferably allocation of the resource reduces a delta between the predicted healthcare event and a proscribed limitation.

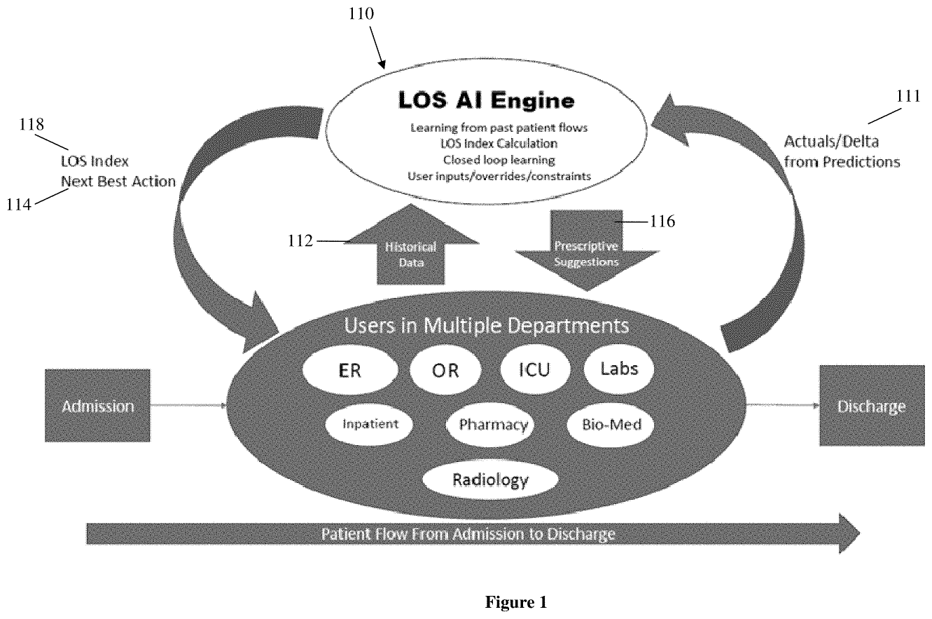

The inventive subject matter contemplates LOS engine 110 as pictorially depicted in FIG. 1. The intent of LOS engine 110 is to learn from historical data 112 as well as to update with real-time intelligence from patients or resources in the healthcare facility. LOS engine 110 provides next best action recommendation 114 and prescriptive suggestions 116 to care teams to minimize bottlenecks and ease patient flow. LOS engine 110 provides continuous guidance to hospital staff during the patient journey from admission to discharge.

Along with providing recommendations and suggestions, LOS engine 110 generates LOS index 118. LOS index 118 is the LOS predicted by LOS engine 110 divided by LOS paid for by Medicare or other payors/insurance providers. Depending on the Diagnosis-related Group (DRG) assigned to an admitted patient, payments from Medicare or other payors to the healthcare facility are fixed and mandated for a certain number of days. Hospital Chief Medical Officers and Case Managers strive to provide efficient care for best clinical outcomes while not falling behind on the LOS Index. LOS engine 110 further receives feedback by way of the delta 111 between the predicted LOS or result and the actual outcome. LOS engine 110 then uses delta 111 to self modify (e.g., change weight of classes of data, change classes of data considered, etc.) to reduce delta 111, preferably to less than 10% or less than 5%.

LOS engine 110 uses various classes of historic data 112 and contemporaneous data from the healthcare facility at least partially sourced from Electronic Health Records or Bed Management Systems, or local or regional demographic data to develop and improve the accuracy of predictions. For example, LOS engine 110 can use any of the following classes of data to train and make predictions: proper bed, level of care; acute care for elderly (ACE) cases; patient population age, diagnosis, sex, payer status, severity, trauma, comorbidities, outliers, increased volume, common or prevalent DRG or diagnosis (e.g., pneumonia, flu, etc.), Better Outcomes for Older adults through Safe Transitions (BOOST), LACE index (length of stay (L), acuity of the admission (A), comorbidity of the patient (C) and emergency department use in the duration of 6 months before admission), past re-admissions, discharge alive, discharge destination; need for healthcare resources (e.g., SNiF bed, diagnostics, mental health resources; need for direct medical equipment (DME) or supplies for home. As LOS engine 110 considers local demographic and social data, it is uniquely able to tune its predictions to the specific ecosystem and environment of any given healthcare facility, for example considering local age of population, local risk factors, local nursing homes, or local skilled nursing facilities.

LOS engine 110 improves hospital-wide patient flow, positively impacts clinical, financial, and operational outcomes, improves revenue, and optimizes staffing and supplies cost.

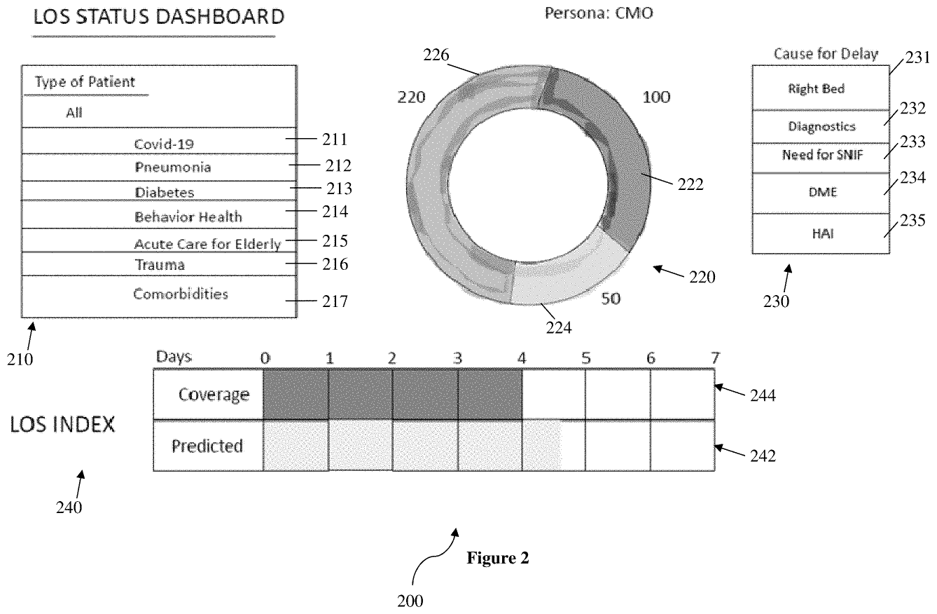

FIG. 2 depicts LOS status dashboard 200, which provides a bird's eye view enabling hospitals to keep tab on LOS of all their patients. Status ring 220 depicts graphically and numerically how many total patients are currently at the hospital (sum of values for bands 222, 224, and 226, here 370 patients), how many patients are low risk or ahead in their LOS index (band 222), how many patients are at a break-even and are medium risk (band 224), and how many patients are high risk and running behind their LOS limit (band 226).

Filter 210 segments the patient population into classes based on higher volume patients, like Covid-19 (class 211), Pneumonia (class 212), Diabetes (class 213), Behavior Health (class 214), ACE (class 215), Trauma (class 216), and Comorbidities (class 217). Filter 230 segments the patient population into classes by cause for delay in patient flow, including lack of right bed (class 231), awaiting diagnostics (class 232), discharge pending dependent on bed at nearby skilled nursing facility (SNiFs) (class 233), discharge pending dependent on DME to go home with the patient (class 234), and Hospital Acquired Infection (HAI) (class 235), for example. As a user selects a class in either filter 210 or 230, LOS index display 240 is updated to display predicted LOS (bar 242) compared to the LOS paid-for according to DRG (bar 244).

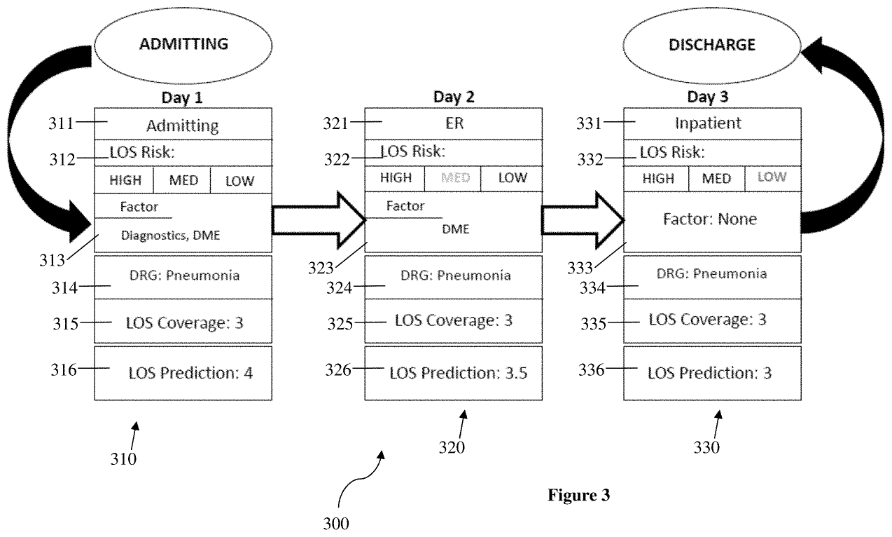

FIG. 3 depicts patient journey tracker 300, which displays daily status for a patient from admitting to discharge. Here, each of arrays 310, 320, and 330 depicts a number of status indicators for day 1 (admitting), day 2, and day 3 (discharge) for the patient, respectively. Each of the arrays depict actionable statuses 311, 321, and 331 (admitting, emergency room, inpatient, respectively), LOS risks 312, 322, and 332 (high, medium, low, respectively), delay factors 313, 323, and 333 (diagnostics and DME, DME, and none, respectively), DRGs 314, 324, and 334 for each day, LOS coverages 315, 325, and 335 applied to each DRG (3 days), and LOS predictions 316, 326, and 336 (4, 3.5, and 3 days, respectively).

Patient journey tracker 300 indicates diagnostics and DME were delay factors on day 1, yielding a LOS prediction that exceeds LOS coverage. By day 2, the LOS engine has recommended proactive steps to resolve the delay factor for diagnostics, but DME remains as a delay factor, yielding a LOS prediction that still exceeds LOS coverage. By day 3, the LOS engine once again has recommended proactive steps to resolve the DME delay, and the patient is set to be discharged. Such a system is a substantial improvement over the art. Significantly, with the rise of Covid-19, hospitals must exercise better capacity management. Accurate prediction of LOS and identifying and resolving related delay factors significant improves hospital response to Covid-19 and future pandemics.

Descriptions throughout this document include information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary. The recitation of ranges of values herein is merely intended to serve as a shorthand method of referring individually to each separate value falling within the range. Unless otherwise indicated herein, each individual value is incorporated into the specification as if it were individually recited herein.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

The discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of implementing a resource management system in a healthcare facility, comprising:
using a first health data to develop a model to predict a first resource requirement;
assessing a predictive ability of the model by applying a second health data to the model to predict a second resource requirement and comparing the second resource requirement to a known resource requirement;
informationally coupling the model with a database of health data for the healthcare facility, wherein the database includes contemporaneous data from the healthcare facility; and
implementing the resource management system comprising the model in the healthcare facility.

2. The method of claim 1, wherein the model comprises a machine learning algorithm.

3. The method of claim 2, wherein the machine learning algorithm employs at least one of supervised, unsupervised, reinforcement, or ensemble learning methodology.

4. The method of claim 1, further comprising applying the model to contemporaneous data from the healthcare facility to predict an unknown resource requirement.

5. The method of claim 1, wherein the resource requirement is at least one of length of stay, required equipment, or required personnel for a patient.

6. The method of claim 1, wherein the model further identifies a class of health data that affects a predicted outcome of the model by more than 10%.

7. The method of claim 1, wherein the database includes classes of data related to an operational outcome of the healthcare facility, a financial outcome of the healthcare facility, a diagnosis of a patient, a prognosis of a patient, a patient outcome, or a treatment of a patient.

8. The method of claim 2, wherein the machine learning algorithm self modifies to minimize a delta between a predicted resource requirement and an actual resource requirement.

9. The method of claim 1, wherein the resource management system further identifies whether a predicted resource requirement exceeds a limit.

10. The method of claim 1, wherein the resource management system further provides a recommended act to minimize a delta between a predicted resource requirement and a limit.

11. The method of claim 1, wherein the resource management system further assigns a risk index to a patient based on a delta between a predicted resource requirement and a limit.

12. A resource management system for a healthcare facility, comprising:
a predictive element informationally coupled to a database of health data regarding the healthcare facility; and
a health data generator coupled to the database;
wherein the health data generator provides a class of contemporaneous health data to the database; and
wherein the predictive element uses the database to make a model to predict a health event in the healthcare facility based on the class of contemporaneous health data.

13. The system of claim 12, wherein the model comprises a machine learning algorithm that employs at least one of supervised, unsupervised, reinforcement, or ensemble learning methodology.

14. The system of claim 12, wherein the health event is at least one of length of stay, required equipment, or required personnel for a patient.

15. The system of claim 12, wherein the class of contemporaneous health data relates to at least one of an operational outcome of the healthcare facility, a financial outcome of the healthcare facility, a diagnosis of a patient, a prognosis of a patient, a patient outcome, or a treatment of a patient.

16. The system of claim 13, wherein the machine learning algorithm self modifies to minimize a delta between a predicted health event and an actual health event.

17. The method of claim 12, wherein the resource management system further provides a recommended act to minimize a delta between a predicted health event and a proscribed limitation.

18. A method of improving resource management in a healthcare facility, comprising:
developing a model to predict a healthcare event;
accessing contemporaneous data regarding the healthcare facility;
applying the model to the contemporaneous data to predict the healthcare event; and
allocating a resource responsive to the healthcare event.

19. The method of claim 18, wherein the model comprises a machine learning algorithm trained on historic data related to the healthcare facility or the healthcare event.

20. The method of claim 18, wherein allocation of the resource reduces a delta between the predicted healthcare event and a proscribed limitation.
